(19) United States
(12) Patent Application Publication
(10) Pub. No.: US 2006/0003428 A1
(43) Pub. Date: Jan. 5, 2006

(54) Enzymatic resolution of an alpha-substituted carboxylic acid or an ester thereof by Carica papaya lipase

(75) Inventor: Shau-Wei Tsai, Tainan City (TW)

Correspondence Address: FOLEY AND LARDNER LLP;SUITE 500, 3000 K STREET NW, WASHINGTON, DC, 20007, US

(73) Assignee: NATIONAL CHENG KUNG UNIVERSITY

(21) Appl. No.: 11/168,490

(22) Filed: Jun. 29, 2005

(30) Foreign Application Priority Data
Jun. 30, 2004 (TW) 093119718

(51) Int. Cl. C12P 7/62

(52) U.S. Cl. 435/135; 435/280

(57) ABSTRACT

Disclosed herein is a process for enzymatically resolving a mixture of R- and S-enantiomers of an α-substituted carboxylic acid or an ester or thioester thereof, in which a Carica papaya lipase is used as a biocatalyst to effect the resolution as desired.

### CROSS-REFERENCE TO RELATED APPLICATION

This application claims the benefits of Taiwan Patent Application No. 093119718, entitled “Method for kinetic resolution of α-substituted acids and esters thereof” and filed on Jun. 30, 2004.

### BACKGROUND OF THE INVENTION

1. Field of the Invention

This invention relates to a process for enzymatically resolving a mixture of R- and S-enantiomers of an α-substituted carboxylic acid or an ester or thioester thereof, in which a Carica papaya lipase is used as a biocatalyst to effect the resolution as desired.

2. Description of the Related Art

Optically active α-substituted carboxylic acids or esters or thioester thereof, are a group of compounds having a stereogenic center at the α-carbon thereof. Enantiomerically enriched α-substituted carboxylic acids, such as α-(hetero)arylcarboxylic acids, α-aryloxypropionic acids, α-alkylcarboxylic acids, α-halogencarboxylic acids and α-amino acids, are of considerable importance as synthetic units in the pharmaceutical and agrochemical sectors or as resolution agents (Sheldon, R. A, “Chirotechnology”, 1993, pp. 205-270; Kazlauskas, R. and Bornscheuer, U., “Biotrasnformations with lipases,” Biotechnology, Vol. 8a, 1998, pp. 103-118; Faber, K., Biotransformations in Organic Chemistry, 2000, pp. 94-123 and pp. 344-366).

For example, α-aryloxypropionic acids, such as commercially available (S)-naproxen, (S)-fenoprofen, (S)-ibuprofen, (S)-ketoprofen, (S)-flurbiprofen and (S)-suprofen, are non-steroid anti-inflammatory drugs (NSAIDs) having of analgesic, antipyretic and anti-inflammatory effects (Chang, C. S. et al., “Lipase-catalyzed dynamic resolution of naproxen 2,2,2-trifluoroethyl thioester by hydrolysis in isooctane,” Biotechnology and Bioengineering, 1999, Vol. 64, pp. 120-126; U.S. Pat. No. 6,201,151 issued to S. -W. Tsai and C. -S. Chang; Sehgal, A. C. and Kelly, R. M., “Strategic selection of hyperthermophilic esterases for resolution of 2-arylpropionic esters,” Biotechnology Progress, 2003, Vol. 19, pp. 1410-1416; Sleenkamp, L. and Brady, D., “Screening of commercial enzymes for the enantioselective hydrolysis of R,S-naproxen ester,” Enzyme and Microbial Technology, 2003, Vol. 32, pp. 472-477; Lin, H. -Y. and Tsai, S. -W., “Dynamic kinetic resolution of (R,S)-naproxen 2,2,2-trifluoroethyl ester via lipase-catalyzed hydrolysis in micro-aqueous isooctane,” Journal of Molecular Catalysis B: Enzymatic, 2003, Vol. 24-25, pp. 111-120).

(R)-α-aryloxypropionic acids, such as (R)-2-phenoxypropionic acid, (R)-2-(4-chlorophenoxy)propionic acid, commercially available (R)-Mecoprop and (R)-Diclofop, etc., may be used as herbicides or an intermediates for the synthesis of herbicides (Ujang, Z. et al., “The kinetic resolution of 2-(4-chlorophenoxy)propionic acid using Candida rugosa lipase,” Process Biochemistry, 2003, Vol. 38, pp. 1483-1488). In addition, (R)-2-halogeno-2-arylacetic acids, such as (R)-2-chloro-2-phenylacetic acid, (R)-2-bromo-o-tolylacetic acid, etc., may serve as an intermediate for the above-described herbicides or pharmaceuticals (Guieysse, D. et al., “Lipase-catallyzed enantioselective transesterification toward esters of 2-bromo-tolyacetic acids,” Tetrahedron: Asymmetry, 2003, Vol. 14, pp. 317-323).

Optically active 2-methylalkanoic acids, such as (S)-2-methylhexanoic acid and (S)-2-methylbutanoic acid, may serve as intermediates for the synthesis of insect pheromones, spices and artificial sweeteners (Heinsman, N. W. J. T. et al., “Lipase-mediated resolution of branched chain fatty acids,” Biocatalysis and Biotransformation, 2002, Vol. 20, pp. 297-309).

Recently, due to the great advancement in enzymatic engineering techniques, there have been established not a few of industrial processes that utilize a highly enantioselective and organic solvent-endurable lipase to effect the hydrolysis, esterification, transesterification or amination resolution on racemates of the aforesaid α-aryl propionic acids, or the corresponding ester, thioester or amide derivatives thereof, in the presence/absence of organic solvent(s). Enzyme-catalyzed resolution of racemic compounds has become a valuable method for obtaining optically pure pharmaceutical, agricultural, and other specialty chemicals.

Lipases (triacylglycerol hydrolases, EC 3.1.1.3) as versatile biocatalysts have been widely applied to lipids conversion and kinetic resolution of a variety of racemic compounds (Kazlauskas and Bomscheuer (1998), supra; K. Faber (2000), supra). Currently, most industrial lipases are generally produced from microorganisms (such as Penicillium sp., Geotrichum sp., Aspergillus sp., Rhizomucor sp., Candida sp. or Pseudomonas sp.) or animals (pancreatic and pregastric tissues of ruminants)(Steenkamp and Brady (2003), supra).

On the basis of the racemic starting substrate, the standard kinetic resolution process has a disadvantage that only at most 50% of the desired optically active product can be obtained thereby. In order to increase the optical purity and the conversion of the product of interest, processes of adding a racemization catalyst, such as a base, an organic metal, a halogen ion or a racemase, into the reaction mixture during the resolution process were further developed, according to which the dynamic kinetic resolution of the resolving enzyme could be greatly facilitated (see U.S. Pat. No. 6,201,151 issued to S. -W. Tsai and C. -S. Chang; C. -Y Chen et al. (2002), J. Org. Chem., Vol. 67, No. 10, pp. 3323-3326).

Specifically, the applicant disclosed in U.S. Pat. No. 6,201,151 a process for preparing an optically active (S)-α-aryl propionic acid or ester or thioester thereof, which may be conducted in different aqueous organic solvents in the presence of an (S)-stereoselective lipase and a base, and an alcohol when needed, to effect the hydrolysis or transesterification of a selected racemic thioester of α-aryl propionic acid, so that the desired (S)-α-aryl propionic acid or ester or thioester thereof can be obtained theoretically at a conversion of approximately 100% and with high optical purity. Lipases suitable for use in said process are derived from microbial origins, including Aspergillus niger, Candida rugosa, Geotrichum, Pseudomonas cepacia, Rhizopus oryzae, etc., and is preferably derived from Candida rugosa.

In contrast to the high enantioselectivity toward alcohols and amines, most lipases show low to moderate enantioselectivity for carboxylic acids (Kazlauskas and Bomscheuer (1998), supra; K. Faber (2000), supra). It is not the case for Candida rugosa lipases (CRL), which exhibit high enantioselectivity for α-arylpropionic acids and α-aryloxypropionic acids, although purification or modification of lipase isoenzymes from the crude preparation is usually imperative before performing the reaction (I. J. Colton et al. (1995), J. Org. Chem., 60:212-217; J. J. Lalonde et al. (1995), J. Am. Chem. Soc., 117:6845-6852). However, in general, CRL shows low tolerances to polar organic solvents, extreme pH, and high temperature. Therefore, selecting or discovering lipases that have high enzymatic activity, enantioselectivity, and stability under a high temperature for chiral acids is clearly a prerequisite for the development of highly competitive industrial bioprocesses.

Although plant lipases seem to be very attractive owing to their low cost, ease of purification and wide availability from natural sources, the low levels of lipase content in the post-germination seeds, bran portion of the grain, and wheat germ have limited their extensive use in pilot or large-scale applications.

Recently, lipases from plant latex, for example, the Caricaceae or Euphorbiaceae latex, have become available in large amounts (C. Dhuique-Mayer et al. (2001), Biotechnol. Left., 23:1021-1024; C. Palocci et al. (2003), Plant Sci., 165:577-582; P. Villeneuve (2003), . Eur. J. Lipid Sci. Technol., 105:308-317). The spray-dried Carica papaya latex, with the commercial name papain, is well known for containing many cysteines thiol-proteases, e.g. papain (EC 34.4.22.2), chmopapains A, B1, B2 and B3 and papaya peptidase II, and others like lysozyme, glutaminyl cyclase, class II chitinase and lipase (Moussaoui AEI et al. (2001), CMLS Cell Mol Life Sci 58:556-570). The lipase activity is located in the non-water-soluble fraction of the latex, suggesting that C. papaya lipase (CPL) is naturally bound and immobilized to the nonsoluble matrix. The crude papain is largely available and cheap, e.g. about one-fourth to one-third price of the crude Candida rugosa lipase (CRL) from Sigma, and hence, has been regarded as a promising alternative to microbial lipases in lipid conversions. However, except for one report mentioning the use of CPL in the kinetic resolution of chiral sn-3 triglycerides (P. Villeneuve et al. (1995), J. Am. Oil Chem., 72:753-755), the properties of CPL in terms of enzymatic activity, substrate selectivity, thermal stability, etc., have yet to be explored.

### SUMMARY OF THE INVENTION

The applicant surprisingly found from experiments that Carica papaya lipase was highly enantioselective to either the S-enantiomer or the R-enantiomer of a selected α-substituted carboxylic acid or an ester or thioester thereof. Therefore, in one aspect, this invention provides a process for enzymatically resolving a mixture of R- and S-enantiomers of an α-substituted carboxylic acid, or an ester or thioester thereof, of formula (I): 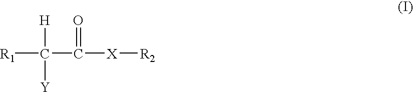
wherein
   X represents —O— or —S—;
   Y is a halogen or a methyl group;
   R₁ represents: a straight-chain or branched saturated or unsaturated C₁-C₂₀ aliphatic group optionally substituted with one to three substituents selected from the group consisting of halo, amino, cyano, hydroxy, —SH, —COOH, —CF₃, —OCF₃, —SCF₃, —CONH₂, a C₁-C₆ alkoxy group and an aryl group; an aryl group, an aryloxy group or a C₃-C₁₂ heterocyclic group containing one to three heteroatoms selected from O, S and N, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N; and
   R₂ represents: H; a straight-chain or branched saturated or unsaturated C₁-C₁₂ aliphatic group optionally substituted with one to three substituents selected from the group consisting of halo, amino, cyano, hydroxy, —CF₃, —OCF₃, —SCF₃, —Si(CH₃)₃, a C₁-C₄ alkyloxy group, a C₁-C₄ alkylthio group, an aryl group, vinyl and a 2-alkenyl group having 3 to 12 carbon atoms; an aryl group or a C₃-C₁₂ heterocyclic group containing one to three heteroatoms selected from O, S and N, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N;
with the proviso that Y and R₁ cannot be methyl at the same time;
the process comprising subjecting the mixture of R- and S-enantiomers of the α-substituted carboxylic acid or ester or thioester thereof of formula (I) to an enzymatic resolution catalyzed by a Carica papaya lipase in a liquid phase.

In a preferred embodiment of the process according to this invention, the mixture comprises R- and S-enantiomers of an α-substituted carboxylic acid ester or thioester of formula (I), and the enzymatic resolution of the mixture by the Carica papaya lipase is conducted in a liquid phase comprising a solvent system selected from an aqueous solution, a water-saturated organic solvent and combinations thereof forming a biphasic solution, such that either R-form or S-form of the α-substituted carboxylic acid ester or thioester of formula (I) is enantioselectively hydrolyzed by the Carica papaya lipase.

In another preferred embodiment of the process according to this invention, the mixture comprises R- and S-enantiomers of an α-substituted carboxylic acid ester or thioester of formula (I), and the enzymatic resolution of the mixture by the Carica papaya lipase is conducted in a liquid phase comprising an anhydrous organic solvent in combination with an alcohol, such that either R-form or S-form of the α-substituted carboxylic acid ester or thioester of formula (I) is enantioselectively transesterified by the Carica papaya lipase using said alcohol.

In a yet preferred embodiment of the process according to this invention, the mixture comprises R- and S-enantiomers of an α-substituted carboxylic acid of formula (I), and the enzymatic resolution of the mixture by the Carica papaya lipase is conducted in a liquid phase comprising an anhydrous organic solvent in combination with an alcohol, such that either R-form or S-form of the α-substituted carboxylic acid of formula (I) is enantioselectively esterified by the Carica papaya lipase using said alcohol.

When conducting the process of this invention as described above, the liquid phase, which preferably comprises an organic solvent system, may additionally comprise an organic base that acts as a racemization catalyst, so as to increase the conversion of the desired optically active products

It has been found that optically active products in high purity and high yield can be more efficiently and economically obtained from the practice of the processes according to this invention, as compared to previous processes using Candida rugosa lipase (CRL) as the biocatalyst.

The above and other objects, features and advantages of this invention will become apparent with reference to the following detailed description of the invention.

### DETAILED DESCRIPTION OF THE INVENTION

Most of the currently available kinetic resolution processes for α-substituted carboxylic acids or esters thereof utilize lipases which are very expensive and difficult to obtain. In order to reduce the cost needed for carrying out such processes and to increase the enantioselectivity of the resolution reaction, the applicant tired to find new lipases that are suitable for use as a biocatalyst in the enzymatic resolution of α-substituted carboxylic acids or esters thereof.

Papaya is a very important economic crop in tropical and subtropical areas in the world. In comparison to lipases of microbial origin, such as Candida rugosa lipase, Carica papaya lipase is comparatively cheap and easy to obtain. The applicant surprising found that Carica papaya lipase can enantioselectively catalyze the hydrolysis, esterification or transesterification of either R-form or S-form of a selected α-substituted carboxylic acid or an ester or thioester thereof, giving the desired optically active product in high yield, high purity and high conversion. In addition, Carica papaya lipase exhibits superior thermal stability and tolerance to various organic solvents.

Therefore, this invention provides a process for enzymatically resolving a mixture of R- and S-enantiomers of an α-substituted carboxylic acid or an ester or thioester thereof of formula (I): 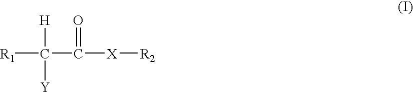
wherein
   X represents —O— or —S—;
   Y is a halogen or a methyl group;
   R₁ represents: a straight-chain or branched saturated or unsaturated C₁-C₂₀ aliphatic group optionally substituted with one to three substituents selected from the group consisting of halo, amino, cyano, hydroxy, —SH, —COOH, —CF₃, —OCF₃, —SCF₃, —CONH₂, a C₁-C₆ alkoxy group and an aryl group; an aryl group, an aryloxy group or a C₃-C₁₂ heterocyclic group containing one to three heteroatoms selected from O, S and N, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N; and
   R₂ represents: H; a straight-chain or branched saturated or unsaturated C₁-C₁₂ aliphatic group optionally substituted with one to three substituents selected from the group consisting of halo, amino, cyano, hydroxy, —CF₃, —OCF₃, —SCF₃, —Si(CH₃)₃, a C₁-C₄ alkyloxy group, a C₁-C₄ alkylthio group, an aryl group, vinyl and a 2-alkenyl group having 3 to 12 carbon atoms; an aryl group or a C₃-C₁₂ heterocyclic group containing one to three heteroatoms selected from O, S and N, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N;
   with the proviso that Y and R₁ cannot be methyl at the same time; the process comprising subjecting the mixture of R- and S-enantiomers of the α-substituted carboxylic acid or ester or thioester thereof of formula (I) to an enzymatic resolution catalyzed by a Carica papaya lipase in a liquid phase.

According to this invention, the Carica papaya lipase may be prepared from a latex exudate of a plant of Carica papaya, e.g., the exuded latex of the leaves, stems, immature fruits or the wounded surfaces of a plant of Carica papaya. A spray-dried Carica papaya latex, with the commercial name papain, is available from Sigma Co. (St. Louis, Mo., USA, product code P3375, a cystine protease of 2.1 units/mg solid, product from Sri Lanka). A partially purified CPL (PCPL) may be obtained by dissolving the commercial papain or a self-prepared Carica papaya latex in an aqueous solution or a buffered solution or an organic solvent with gentle stirring, followed by centrifugation or filtration, to give a precipitate, which is subsequently lyophilized. The resultant lyophilized product is ready for use or may be subjected to the above treatments again so as to give a more pure enzyme product.

According to this invention, the term “aliphatic group” as used herein includes straight-chain or branched saturated or unsaturated alkyl groups, alkenyl groups, alkynyl groups and cycloalkyl groups, each of which may be optionally substituted with one to three substituents as described for the R₁ and R₂ groups.

According to this invention, the term “aryl group” as used herein Includes phenyl, phenoxy, naphthyl, naphthoxy, tetrahydronaphthyl, etc., each of which may be optionally substituted with one to three substituents as described for the R₁ and R₂ groups.

According to this invention, the term “heterocyclic group” as used herein includes thienyl, thenoyl, furyl, pyridyl, pyrazinyl, imidazyl, pyranyl, etc., each of which may be optionally substituted with one to three substituents as described for the R₁ and R₂ groups.

In a preferred embodiment of this invention, the R₁ group on the α-substituted carboxylic acid or an ester or thioester thereof of formula (I) is selected from the group consisting of a straight-chain or branched C₁-C₂₀ alkyl group, a straight-chain or branched C₁-C₂₀ alkenyl group, a straight-chain or branched C₁-C₂₀ alkynyl group, and a straight-chain or branched C₁-C₂₀ cycloalkyl group, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N.

In another preferred embodiment of this invention, the R₁ group on the α-substituted carboxylic acid or an ester or thioester thereof of formula (I) is selected from the group consisting of an aryl group and a C₃-C₁₂ heterocyclic group containing a heteroatom selected from N, O and S, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N.

Representative examples of the R₁ group include, but are not limited to, butyl, hexyl, octyl, pent-3-enyl, phenyl, phenoxy, 2-chlorophenyl, benzyl, phenylethyl, naphthyl, 6-methoxy-2-naphthyl, naphthoxy, (2-fluoro-3-phenyl)phenyl, 4-chlorophenoxy, 2-(2,4-dichlorophenoxy)phenyl, m-phenoxy-phenyl, p-phenoxy-phenyl, 4-isobutyl-phenyl, (2-benzoyl)phenyl, p-thenoyl-phenyl, N-methylimidazyl, 4-nitropyridyl, pyrazinyl, etc.

Preferably, the R₂ group has at least one electron-withdrawing substituent positioned on the C2 and/or C3 position thereof.

In a preferred embodiment of this invention, the R₂ group on the α-substituted carboxylic acid or an ester or thioester thereof of formula (I) is hydrogen.

In another preferred embodiment of this invention, the R₂ group on the α-substituted carboxylic acid or an ester or thioester thereof of formula (I) is selected from the group consisting of a straight-chain or branched C₁-C₁₂ alkyl group, a straight-chain or branched C₁-C₁₂ alkenyl group, a straight-chain or branched C₁-C₁₂ alkynyl group, and a straight-chain or branched C₁-C₁₂ cycloalkyl group, each group being optionally substituted with one to three substituents selected from the group consisting of halo, amino, cyano, hydroxy, —CF₃, —OCF₃, —SCF₃, —Si(CH₃)₃, a C₁-C₄ alkyloxy group, a C₁-C₄ alkylthio group, an aryl group, vinyl and a 2-alkenyl group having 3 to 12 carbon atoms.

In a yet preferred embodiment of this invention, the R₂ group on the α-substituted carboxylic acid or an ester or thioester thereof of formula (I) is selected from the group consisting of an aryl group or a C₃-C₁₂ heterocyclic group containing a heteroatom selected from N, O and S, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N.

Representative examples of the R₂ group in the α-substituted carboxylic acid or an ester thereof of formula (I) include, but are not limited to, methyl, ethyl, propyl, butyl, pentyl, hexyl, heptyl, octyl, nonyl, decyl, vinyl, ethynyl, 2-allyl, 2-butenyl, 2-chloroethyl, 2-bromoethyl, 2,2,2-trifluoroethyl, 2,2,2-trichloroethyl, 2,2,2-tribromoethyl, 2-chloropropyl, (trimethylsilyl)methyl, (trimethylsilyl)ethyl, benzyl, naphthylmethyl, etc.

Representative examples of the α-substituted carboxylic acid ester or thioester of formula (I) include, but are not limited to, the following compounds: an ethyl, propyl, butyl, hexyl, phenyl or trifluoroethyl ester of naproxen, fenoprofen, ibuprofen, ketoprofen, suprofen, flurbiprofen, 2-phenyl propionic acid, 2-(4-chlorophenoxy)propionic acid or 2-chloro-2-phenylacetic acid; an ethyl, propyl, butyl, hexyl, phenyl or trifluoroethyl thioester of naproxen, fenoprofen, ibuprofen, ketoprofen, suprofen, flurbiprofen, 2-phenyl propionic acid, 2-(4-chlorophenoxy)propionic acid or 2-chloro-2-phenylacetic acid; and diclofog methyl ester.

Representative examples of the α-substituted carboxylic acid of formula (I) include, but are not limited to, the following compounds: naproxen, fenoprofen, ibuprofen, ketoprofen, suprofen, flurbiprofen, 2-phenyl propionic acid, 2-(4-chlorophenoxy)propionic acid, 2-chloro-2-phenylacetic acid, and diclofog.

The process of this invention may be conducted in a liquid phase comprising a solvent system selected from an aqueous solution, an anhydrous organic solvent, an organic solvent saturated with water, and combinations thereof forming a biphasic solution.

Aqueous solutions suitable for use in the process of this invention may be selected from water and buffered aqueous solutions.

Organic solvent suitable for use in the process of this invention may be selected from isooctane, heptane, hexane, cyclohexane, pentane, decane, toluene, benzene, carbon tetrachloride, t-butanol, t-pentanol, isopropyl ether, methyl t-butyl ether, methyl isobutyl ether, and combinations thereof.

Alternatively, the process of this invention may be conducted in a liquid phase comprising a biphasic solution constituted of an aqueous solution and one or more organic solvents that form a miscible organic phase.

The process of this invention may be used for the enzymatic resolution of a racemic mixture of an α-substituted carboxylic acid ester or thioetser of formula (I). A mixture of an α-substituted carboxylic acid ester or thioetser of formula (I), which contains excessive R-enantiomer or S-enantiomer, may also be treated by the process of this invention.

In the process of this invention, an organic base, which acts as a racemization catalyst, may be added in the liquid phase so as to increase the conversion of the desired optically active products.

Organic base suitable for use in the process of this invention may be selected from the group consisting of tertiary amines, amidines, guanidines, phosphazene bases, and combinations thereof. Preferably, the organic base is selected from the group consisting of triethylamine, tributylamine, trioctylamine, 1-t-butyl-4,4,4-tris(dimethylamino)-2,2-bis[tris(dimethylamino)-phosphoranylidenamino]-2λ⁵,4λ⁵-catenadi(phosphazene), diethylaminomethyl-polystyrene, t-butylimino-tris(dimethylamino)phosphorane, 7-methyl-1,5,7-triazabicyclo[4,4,0]dec-5-ene, t-butylimino-tris(pyrrolidino)phosphorane, 1,8-diazabicyclo[5,4,0]undec-7-ene, 1,4-diazabicyclo[2.2.2]octane, and combinations thereof. In addition, the organic base may be carried on a support selected from an organic support and an inorganic support. For example, the organic base is carried on an anion-exchange resin.

The process of this invention may be conducted at a temperature suitable for the Carica papaya lipase to catalyze the enzymatic resolution of the mixture comprising R- and S-enantiomers of a selected α-substituted carboxylic acid ester or thioester of formula (I). Preferably, the process of this invention is conducted at a temperature ranging from 20° C. to 90° C., and more preferably at a temperature ranging from 30° C. to 70° C.

In a preferred embodiment of the process according to this invention, the mixture comprises R- and S-enantiomers of an α-substituted carboxylic acid ester or thioester of formula (I), and the enzymatic resolution of the mixture by the Carica papaya lipase is conducted in a liquid phase comprising a solvent system selected from an aqueous solution, a water-saturated organic solvent and combinations thereof forming a biphasic solution, such that either R-form or S-form of the α-substituted carboxylic acid ester or thioester of formula (I) is enantioselectively hydrolyzed by the Carica papaya lipase.

The hydrolysis of the mixture comprising R- and S-enantiomers of an α-substituted carboxylic acid ester or thioester of formula (I) by the Carica papaya lipase may be schematically expressed by the following scheme.

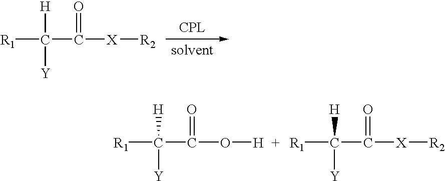

In addition, an organic base as described may be added into the liquid phase, which preferably comprises an organic solvent, so as to facilitate the conversion of the desired optically active R- or S-α-substituted carboxylic acid.

In another preferred embodiment of the process according to this invention, the mixture comprises R- and S-enantiomers of an α-substituted carboxylic acid ester or thioester of formula (I), and the enzymatic resolution of the mixture by the Carica papaya lipase is conducted in a liquid phase comprising an anhydrous organic solvent in combination with an alcohol, such that either R-form or S-form of the α-substituted carboxylic acid ester or thioester of formula (I) is enantioselectively transesterified by the Carica papaya lipase using said alcohol.

The transesterification of the mixture comprising R- and S-enantiomers of an α-substituted carboxylic acid ester or thioester of formula (I) by the Carica papaya lipase may be schematically expressed by the following scheme.

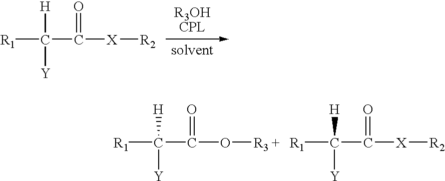

In a yet preferred embodiment of the process according to this invention, the mixture comprises R- and S-enantiomers of an α-substituted carboxylic acid of formula (I), and the enzymatic resolution of the mixture by the Carica papaya lipase is conducted in a liquid phase comprising an anhydrous organic solvent in combination with an alcohol, such that either R-form or S-form of the α-substituted carboxylic acid of formula (I) is enantioselectively esterified by the Carica papaya lipase using said alcohol.

The esterification of the mixture comprising R- and S-enantiomers of an α-substituted carboxylic acid of formula (I) by the Carica papaya lipase may be schematically expressed by the following scheme.

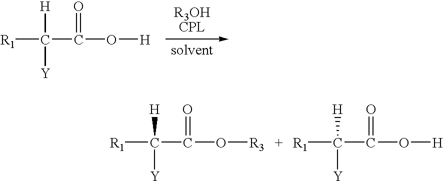

Alcohols suitable for use in the enzymatic resolution of the mixture catalyzed by the Carica papaya lipase is of formula R₃OH, wherein R₃ differs from R₂ and represents: a straight-chain or branched saturated or unsaturated C₁-C₁₂ aliphatic group optionally substituted with one to three substituents selected from the group consisting of halo, amino, cyano, hydroxy, —CF₃, —OCF₃, —SCF₃, —Si(CH₃)₃, a C₁-C₄ alkyloxy group, a C₁-C₄ alkylthio group, an aryl group, vinyl and a 2-alkenyl group having 3 to 12 carbon atoms; an aryl group or a C₃-C₁₂ heterocyclic group containing one to three heteroatoms selected from O, S and N, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a 03-C₁₋₂ heterocyclic group containing one to three heteroatoms selected from O, S and N.

Preferably, the alcohol is selected from the group consisting of propanol, butanol, hexanol, trimethylsilyl methanol, and 2-N-morpholinoethanol.

This invention will be further described by way of the following examples. One of ordinary skill in the art is familiar with many techniques and teachings allowing the modification of these examples and the examples noted throughout this disclosure that would also employ the basic, novel, or advantageous characteristics of the invention. Thus, the scope of this invention is not limited by the particular examples listed here or elsewhere.

### EXAMPLES

I. Materials:

1. Isooctane, cyclohexane, isopropanol and acetic acid glacial were purchased from Tedia Co. (Fairfield, Ohio, USA);
2. Trioctylamine and 2,2,2-trifluoroethanol were purchased from Aldrich Co. (Milwaukee, Wis., USA);
3. Trimethylsilyl methanol was purchased from Fluka Co. (Buchs, Switzerland);
4. Racemic (R,S)-naproxen was obtained by racemizing (S)-naproxen (Sigma Co., St. Louis, Mo., USA) at 140° C. in ethylene glycol containing NaOH(S. -W Tsai and H. -J. Wei (1994), Enzyme Microb. Technol. 16:328-333)
5. Racemic (R,S)-naproxen 2,2,2-trifluoroethyl thioester was obtained by racemizing (S)-naproxen (Sigma Co., St. Louis, Mo., USA), followed by esterifying the resultant racemic (R,S)-naproxen with 2,2,2-trifluoroethanethiol (C. -Y. Chen (2002), J. Org. Chem., 67 (10): 3323-3326);
6. Racemic (R,S)-naproxen 2,2,2-trifluoroethyl ester was obtained by racemizing (S)-naproxen (Sigma Co., St. Louis, Mo., USA), followed by esterifying the resultant racemic (R,S)-naproxen with 2,2,2-trifluoroethanol (H. -Y Lin and S. -W. Tsai (2003), J. Mol. Catal. B: Enz., 24:111-20);
7. Racemic (R,S)-fenoprofen 2,2,2-trifluoroethyl thioester was obtained by esterifying racemic (R,S)-fenoprofen calcium salt (Sigma Co., St. Louis, Mo., USA) with 2,2,2-trifluoroethanethiol;
8. Racemic (R,S)-ibuprofen 2,2,2-trifluoroethyl thioester was obtained by esterifying racemic (R,S)-ibuprofen (Sigma Co., St. Louis, Mo., USA) with 2,2,2-trifluoroethanethiol;
9. Racemic (R,S)-2-phenyl propionic 2,2,2-trifluoroethyl thioester was obtained by esterifying racemic (R,S)-2-phenyl propionic acid (Sigma Co., St. Louis, Mo., USA) with 2,2,2-trifluoroethanethiol;
10. Racemic (R,S)-diclofog methyl ester was purchased from Riedel-de Haën Co. (Seelve, Germany);
11. Racemic (R,S)-2-chloro-2-phenylacetic 2,2,2-trifluoroethyl thioester was obtained by esterifying 2-chlorophenylacetyl chloride with 2,2,2-trifluoroethanethiol;
12. Racemic (R,S)-2-(4-chlorophenoxy)propionic acid was purchased from Sigma Co. (St. Louis, Mo., USA);
13. Carica papaya lipase (CPL).
   (1) crude papain, which was a commercial product available from Sigma Co. (St. Louis, Mo., USA, product code P3375, a cystine protease of 2.1 units/mg solid, product from Sri Lanka);
   (2) partially purified CPL (PCPL), which was prepared as follows: 1.35 g of the crude papain was added into 15 mL deionized water at 4° C. with gentle stirring for 30 min. The resultant solution was centrifuged at 12,000 rpm for 10 min.
      After discarding the supernatant, the above procedures were repeated to give a precipitate, which was then lyophilized at −40° C. and 100 mmHg for 4 hrs, giving a 15% recovery of PCPL based on the weight of crude papain;
14. Other chemicals of analytical grade that were commercially available are as follows: n-hexane, n-propanol, n-butanol, n-hexanol, 1,2-dimethoxyethane, anhydrous pyridine, phenyl dichlorophosphate, chloroform, sodium chloride, sodium hydroxide, magnesium sulfate, ethyl acetate, etc.
   II. General Procedures:
   1. Preparation of a Water-Saturated Organic Solvent:

A suitable amount of deionized water was added into a selected organic solvent, such as isooctane and cyclohexane. After stirring for a period over 24 hrs, the organic layer was collected for subsequent use. The preparation of the water-saturated organic solvent is preferably performed at a temperature identical to that for carrying out the enzymatic resolution catalyzed by Carica papaya lipase,

2. Synthesis of (R,S)-naproxen 2,2,2-trifluoroethyl Thioester:

To 25 mL of ice-cooled anhydrous 1,2-dimethoxyethane was added 4.30 mmol of (R,S)-naproxen, 1.15 mL of anhydrous pyridine, 1.07 mmol of phenyl dichlorophosphate, and 1000 mg of 2,2,2-trifluoroethanethiol. The resultant mixture was allowed to react at room temperature for 16 hrs with stirring, followed by addition of 20 mL of 1 M ice-cooled NaOH solution. Thereafter, the resultant mixture was added with 25 mL of chloroform with stirring for 30 min so as to extract the product. The organic layer was collected and washed in sequence twice with 50 mL of 1 M NaOH solution and twice with 50 mL of saturated NaCl solution, dried over MgSO₄ for 24 hrs, filtered, and concentrated in vacuo. The resultant oil was purified by silica-gel liquid chromatography with a mobile phase of n-hexane:ethyl acetate (5:1, v/v) and then concentrated in vacuo, giving a white solid product of 62% yield based on the initial (R,S)-naproxen.

Other (R,S)-profen 2,2,2-trifluoroethyl thioesters used in the following examples were prepared in a similar manner, while (R,S)-profen 2,2,2-trifluoroethyl esters were prepared according to the procedures set forth in H. -Y. Lin and S. -W. Tsai (2003), J. Mol. Catal. B: Enz, 24:111-20.

3. High Performance Liquid Chromatography (HPLC) Analysis:

Hydrolysis of (R,S)-naproxen 2,2,2-trifluoroethyl esters in a selected water-saturated organic solvent and esterification of (R,S)-naproxen by n-propanol were monitored by HPLC using a chiral column (S,S)-WHELK-01 purchased from Regis Co. (Morton Grove, Ill., USA) capable of separating the internal standard of 2-nitrotoluene, (R)- and (S)-naproxens, and (R)- and (S)-naproxen esters. The mobile phase was a mixture of n-hexane/isopropanol/acetic acid glacial (80:20:0.5, v/v) at a flow rate of 1.0 mL/min. UV detection at 270 nm was performed for quantification at the column temperature of 25° C.

Hydrolysis of 2,2,2-trifluoroethyl thioesters of different (R,S)-profens, 2-phenyl propionic acid and 2-chloro-2-phenylacetic acid, and esterification of 2-(4-chlorophenoxy)propionic acid with a selected alcohol were monitored by HPLC using a chiral column (Chiralcel OD or OJ-H, Daicel Chemical Industries, Tokyo, Japan) capable of separating the internal standard of nitrotoluene, (R)-and (S)-thioesters or (R)-and (S)-esters, and (R)- and (S)-profens. The mobile phase was a mixture of n-hexane/isopropanol/acetic acid glacial at a flow rate of 1 mL/min. UV detection at 270 nm was performed for quantification at the column temperature of 25° C.

### Example 1

### Kinetic Resolution of Racemic (RS)-naproxen 2,2,2-trifluoroethyl Ester by Enzymatic Hydrolysis using Carica papaya Lipase

A water-saturated organic solvent was prepared using either isooctane or cyclohexane according to the procedures set forth in the preceding section of General procedures II.1.

Racemic (R,S)-naproxen 2,2,2-trifluoroethyl ester was added to the thus-prepared organic solvent to a concentration of 3 mM. To 15 mL of the thus-obtained racemic (R,S)-naproxen ester solution was added with either crude papain (75 mg) or partially purified Carica papaya lipase (PCPL, 11.3 mg). The resultant mixture was allowed to react with stirring under a selected temperature ranging from 35° C. to 70° C. for a predetermined period of time.

Aliquots (200 μL) of samples were taken at predetermined time intervals and subjected to HPLC analysis using a (S,S)-WHELK-01 column (Regis Co., Morton Grove, Ill., USA). The mobile phase was a mixture of n-hexane/isopropanol/acetic acid glacial (80:20:0.5, v/v) at a flow rate of 1.0 mL/min. UV detection at 270 nm was performed for quantification at the column temperature of 25° C.

The time-course variations of the conversion of (S)-naproxen ester at t time (expressed as X_{S}), the conversion of (R)-naproxen ester at t time (expressed as X_{R}), the conversion of racemic (R,S)-naproxen ester at t time (expressed as Xₜ and the optical purity of the product (expressed as eeₚ) were calculated based on the following equations, respectively:
X_{S}=1−(S_{S})ₜ/(S_{S})₀
X_{R}=1−(S_{R})ₜ/(S_{R})₀
Xₜ=1−[(S_{S})ₜ+(S_{R})ₜ]/[(S_{S})₀+(S_{R})₀]
eeₚ=|(X_{S}−X_{R})/(X_{S}+X_{R})|
in which:
(S_{S})₀: initial (S)-naproxen ester concentration (mM) at 0 time;
(S_{R})₀: initial (R)-naproxen ester concentration (mM) at 0 time;
(S_{S})ₜ: (S)-naproxen ester concentration (mM) at t time (hr); and
(S_{R})ₜ: (R)-naproxen ester concentration (mM) at t time (hr).

In addition, the enantiomeric ratio (expressed as E) was defined as the initial reaction rate of (S)-naproxen ester to that of (R)-naproxen ester, or vice versa.

When a racemic mixture of R- and S-enantiomers of an α-substituted carboxylic acid or an ester or thioester thereof of formula (I) is used as the enzyme substrate, then (S_{S})₀=(S_{R})₀, and
Xₜ=1−[(S_{S})ₜ+(S_{R})ₜ]/[(S_{S})₀+(S_{R})₀]=(X_{S}+X_{R})/2

Table 1 summarized the experimental data collected from experiments conducted under different temperatures for a predetermined time interval using different solvent systems and enzymes.

TABLE 1

| | Solvent | Temp. | Time | | | | |
|---|---|---|---|---|---|---|---|
| Enzyme | system | (° C.) | (hrs) | X_{S}(%) | Xₜ(%) | eeₚ(%) | E |
| Curde | Water- | 35 | 5 | 38.3 | | >99 | 778 |
| papain | saturated | 45 | 5 | 46.1 | | >99 | 363 |
| (75 | isooctane | 50 | 5 | 46.4 | | >97 | 194 |
| mg) | | 60 | 5 | 51.7 | | >95 | 122 |
| | | 70 | 3 | 50.7 | | >93 | 32 |
| | Water- | 45 | 120 | 90.2 | 47.6 | >89 | 47 |
| | saturated | | | | | | |
| | cyclohexane | | | | | | |
| PCPL | Water- | 35 | 5 | 39.0 | | >98 | 606 |
| (11.3 | saturated | 45 | 6 | 43.2 | | >97 | 200 |
| mg) | isooctane | 60 | 5 | 44.3 | | >92 | 131 |
| | | 70 | 7 | 59.2 | | >88 | 117 |

### Example 2

### Kinetic Resolution of Racemic (R,S)-naproxen 2,2,2-trifluoroethyl Thioesters by Enzymatic Hydrolysis using Carica papaya Lipase

According to the procedures set forth in the above Example 1, racemic (R,S)-naproxen 2,2,2-trifluoroethyl thioester was added to a selected water-saturated organic solvent to a concentration of 1 mM. To 15 mL of the thus-obtained racemic (R,S)-naproxen thioester solution was added with either crude papain (1350 mg) or partially purified Carica papaya lipase (PCPL, 203 mg). The resultant mixture was allowed to react with stirring under a selected temperature ranging from 35° C. to 60° C. for a predetermined period of time.

Aliquots (200 μL) of samples were taken at predetermined time intervals and subjected to HPLC analysis using a Chiralcel OD column (Daicel Chemical Industries, Tokyo, Japan). The mobile phase was a mixture of n-hexane/isopropanol/acetic acid glacial (97:3:1, v/v) at a flow rate of 1.0 mL/min. UV detection at 270 nm was performed for quantification at the column temperature of 25° C.

The time-course variations of the conversion of (S)-naproxen thioester at t time (expressed as X_{S}), the conversion of (R)-naproxen thioester at t time (expressed as X_{R}), the conversion of racemic (R,S)-naproxen thioester at t time (expressed as Xₜ), the optical purity of the product (expressed as eeₚ) and the E value were calculated according to the descriptions set forth in the above Example 1.

Table 2 summarized the experimental data collected from experiments conducted under different temperatures for a predetermined time interval using different solvent systems and enzymes.

TABLE 2

| | Solvent | Temp, | Time | X_{S} | Xₜ | eeₚ | |
|---|---|---|---|---|---|---|---|
| Enzyme | system | (° C.) | (hrs) | (%) | (%) | (%) | E |
| Curde | Water- | 35 | 240 | 70.1 | | >98 | 298 |
| papain | saturated | 45 | 297 | 79.3 | 45.3 | >97 | 130 |
| (1350 | isooctane | 55 | 240 | 72.2 | | >94 | 64 |
| mg) | | 60 | 240 | 87.0 | | >91 | 48 |
| | | 65 | 240 | 50.4 | | >86 | 18 |
| | Water- | 45 | 240 | 61.2 | 40.3 | >91 | 49 |
| | saturated | | | | | | |
| | cyclohexane | | | | | | |
| PCPL | Water- | 35 | 126 | 66.9 | | >98 | 251 |
| (203 | saturated | 45 | 120 | 88.5 | 45.0 | >97 | 158 |
| mg) | isooctane | 55 | 150 | 94.2 | | >91 | 56 |
| | | 60 | 150 | 96.5 | | >88 | 52 |
| | | 65 | 108 | 96.8 | | >85 | 44 |
| | Water- | 45 | 120 | 77.2 | 40.5 | >93 | 41 |
| | saturated | | | | | | |
| | cyclohexane | | | | | | |

### Example 3

### Kinetic Resolution of Racemic (R,S)-fenoprofen 2,2,2-trifluoroethyl Thioester by Enzymatic Hydrolysis using Carica papaya Lipase

According to the procedures set forth in the above Example 11 racemic (R,S)-fenoprofen 2,2,2-trifluoroethyl thioester was added to a water-saturated isooctane to a concentration of 1 mM. To 15 mL of the thus-obtained racemic (R,S)-fenoprofen thioester solution was added with partially purified Carica papaya lipase (PCPL, 203 mg). The resultant mixture was allowed to react with stirring at 60° C. for a period of 170 hrs. Aliquots (200 μL) of samples were taken and subjected to HPLC analysis using a Chiralcel OD column (Daicel Chemical Industries, Tokyo, Japan). The mobile phase was a mixture of n-hexane/isopropanol/acetic acid glacial (100:1.0:0.5, v/v) at a flow rate of 1.0 mL/min. UV detection at 270 nm was performed for quantification at the column temperature of 25° C.

The time-course variations of the conversion of (S)-fenoprofen thioester at t time (expressed as X_{S}), the conversion of (R)-fenoprofen thioester at t time (expressed as X_{R}), the conversion of racemic (R,S)-fenoprofen thioester at t time (expressed as Xₜ), the optical purity of the product (expressed as eeₚ) and the E value were calculated according to the descriptions set forth in the above Example 1.

The experimental data collected from this example were summarized in Table 3. It can be seen that Carica papaya lipase catalyzed the hydrolysis of (R)-fenoprofen thioester instead of (S)-fenoprofen thioester.

### Example 4

### Kinetic Resolution of Racemic (R,S)-ibuprofen 2,2,2-trifluoroethyl Thioester by Enzymatic Hydrolysis using Carica papaya Lipase

According to the procedures set forth in the above Example 1, racemic (RS)-ibuprofen 2,2,2-trifluoroethyl thioester was added to a water-saturated isooctane to a concentration of 1 mM. To 15 mL of the thus-obtained racemic (R,S)-ibuprofen thioester solution was added with partially purified Carica papaya lipase (PCPL, 203 mg). The resultant mixture was allowed to react with stirring at 45° C. for a period of 104 hrs. Aliquots (200 μL) of samples were taken and subjected to HPLC analysis using a Chiralcel OD column (Daicel Chemical Industries, Tokyo, Japan). The mobile phase was a mixture of n-hexane/isopropanol (100:0, v/v) at a flow rate of 1.0 mL/min. UV detection at 270 nm was performed for quantification at the column temperature of 25° C.

The time-course variations of the conversion of (S)-ibuprofen thioester at t time (expressed as X_{S}), the conversion of (R)-ibuprofen thioester at t time (expressed as X_{R}), the conversion of racemic (R,S)-ibuprofen thioester at t time (expressed as Xₜ), the optical purity of the product (expressed as eeₚ) and the E value were calculated according to the descriptions set forth in the above Example 1.

The experimental data collected from this example were summarized in Table 3.

### Example 5

### Kinetic Resolution of Racemic (R,S)-2-phenyl Propionic 2,2,2-trifluoroethyl Thioester by Enzymatic Hydrolysis using Carica papaya Lipase

According to the procedures set forth in the above Example 1, racemic (R,S)-2-phenyl propionic 2,2,2-trifluoroethyl thioester was added to a water-saturated isooctane to a concentration of 1 mM. To 15 mL of the thus-obtained racemic (R,S)-2-phenyl propionic thioester solution was added with partially purified Carica papaya lipase (PCPL, 203 mg). The resultant mixture was allowed to react with stirring at 45° C. for a period of 170 hrs. Aliquots (200 μL) of samples were taken and subjected to HPLC analysis using a Chiralcel OD column (Daicel Chemical Industries, Tokyo, Japan). The mobile phase was a mixture of n-hexane/isopropanol/acetic acid glacial (100:0.35:0.22, v/v) at a flow rate of 1.0 mL/min. UV detection at 270 nm was performed for quantification at the column temperature of 25° C.

The time-course variations of the conversion of (S)-2-phenyl propionic thioester at t time (expressed as X_{S}), the conversion of (R)-2-phenyl propionic thioester at t time (expressed as X_{R}), the conversion of racemic (R,S)-2-phenyl propionic thioester at t time (expressed as Xₜ), the optical purity of the product (expressed as eeₚ) and the E value were calculated according to the descriptions set forth in the above Example 1.

The experimental data collected from this example were summarized in Table 3.

### Example 6

### Kinetic Resolution of Racemic (R,S)-diclofog Methyl Ester by Enzymatic Hydrolysis using Carica papaya Lipase

According to the procedures set forth in the above Example 1, racemic (R,S)-diclofog methyl ester was added to a water-saturated isooctane to a concentration of 1.5 mM. To 15 mL of the thus-obtained racemic (R,S)-diclofog methyl ester solution was added with partially purified Carica papaya lipase (PCPL, 15 mg). The resultant mixture was allowed to react with stirring at 45° C. for a period of 18.2 hrs. Aliquots (200 μL) of samples were taken and subjected to HPLC analysis using a Chiralcel OJ-H column (Daicel Chemical Industries, Tokyo, Japan). The mobile phase was a mixture of n-hexane/isopropanol/acetic acid glacial (97:3:1, v/v)) at a flow rate of 1.0 mL/min. UV detection at 270 nm was performed for quantification at the column temperature of 25° C.

The time-course variations of the conversion of (S)-diclofog methyl ester at t time (expressed as X_{S}), the conversion of (R)-diclofog methyl ester at t time (expressed as X_{R}), the conversion of racemic (R,S)-diclofog methyl ester at t time (expressed as Xₜ), the optical purity of the product (expressed as eeₚ) and the E value were calculated according to the descriptions set forth in the above Example 1.

The experimental data collected from this example were summarized in Table 3. It can be seen that Carica papaya lipase catalyzed the hydrolysis of (R)-diclofog methyl ester instead of (S)-diclofog methyl ester.

### Example 7

### Kinetic Resolution of Racemic (R,S)₂-chloro-2-phenylacetic 2,2,2-trifluoroethyl Thioester by Enzymatic Hydrolysis using Carica papaya Lipase

According to the procedures set forth in the above Example 1, racemic (R,S)-2-chloro-2-phenylacetic 2,2,2-trifluoroethyl thioester was added to a water-saturated isooctane to a concentration of 1 mM. To 15 mL of the thus-obtained racemic (R,S)-2-chloro-2-phenylacetic thioester solution was added with partially purified Carica papaya lipase (PCPL, 25 mg). The resultant mixture was allowed to react with stirring at 45° C. for a period of 48 hrs. Aliquots (200 μL) of samples were taken and subjected to HPLC analysis using a Chiralcel OJ-H column (Daicel Chemical Industries, Tokyo, Japan). The mobile phase was a mixture of n-hexane/isopropanol/acetic acid glacial (240:10:1, v/v)) at a flow rate of 1.0 mL/min. UV detection at 240 nm was performed for quantification at the column temperature of 25° C.

The time-course variations of the conversion of (S)-2-chloro-2-phenylacetic thioester at t time (expressed as X_{S}), the conversion of (R)-2-chloro-2-phenylacetic thioester at t time (expressed as X_{R}), the conversion of racemic (R,S)-2-chloro-2-phenylacetic thioester at t time (expressed as Xₜ), the optical purity of the product (expressed as eeₚ) and the E value were calculated according to the descriptions set forth in the above Example 1.

The experimental data collected from this example were summarized in Table 3. It can be seen that Carica papaya lipase catalyzed the hydrolysis of (R)-2-chloro-2-phenylacetic thioester instead of (S)-2-chloro-2-phenylacetic thioester.

TABLE 3

| | Temp. | Time | X_{R} or X_{S} | Xₜ | eeₚ | |
|---|---|---|---|---|---|---|
| Example | (° C.) | (hrs) | (%) | (%) | (%) | E |
| 3 | 60 | 170 | X_{R} = 90.0 | 50.5 | >78.2 | 57 |
| 4 | 45 | 104 | X_{S} = 23.3 | 14.8 | 57.4 | 3 |
| 5 | 45 | 170 | X_{S} = 29.0 | 16.5 | 75.8 | 8 |
| 6 | 45 | 18.2 | X_{R} = 53.9 | 30.1 | >90 | 13 |
| 7 | 45 | 48 | X_{R} = 71.9 | 58.4 | >23 | 2.1 |

### Example 8

### Kinetic Resolution of (R,S)-naproxen 2,2,2-trifluoroethyl Ester by Enzymatic Hydrolysis using Carica papaya Lipase in the Presence of Trioctylamine

According to the procedures set forth in the above Example 1, racemic (R,S)-naproxen 2,2,2-trifluoroethyl thioester was added to a water-saturated isooctane to a concentration of 1 mM. Aliquots (10 mL) of the thus-obtained racemic (R,S)-naproxen 2,2,2-trifluoroethyl thioester solution were added with partially purified Carica papaya lipase (PCPL, 135 mg) and trioctylamine in different concentrations, respectively. The resultant mixtures were allowed to react with stirring at 45° C. for a predetermined period of time. Thereafter, aliquots (200 μL) of samples were taken and subjected to the HPLC analysis as described in the above Example 2

The time-course variations of the conversion of (S)-naproxen thioester at t time (expressed as X_{S}), the conversion of (R)-naproxen thioester at t time (expressed as X_{R}), the conversion of racemic (R,S)-naproxen thioester at t time (expressed as Xₜ), and the optical purity of the product (expressed as eeₚ) were calculated according to the descriptions set forth in the above Example 1.

The experimental data collected from this example were summarized in Table 4. It can be seen that the addition of trioctylamine lead to an increase of the eeₚ value up to approximately 100%.

TABLE 4

| Concentration of | Time | Xₜ | eeₚ |
|---|---|---|---|
| trioctylamine (mM) | (hrs) | (%) | (%) |
| 0 | 123 | 44 | 95 |
| 19 | 120 | 55 | ˜100 |
| 59 | 120 | 70 | ˜100 |
| 99 | 120 | 77 | ˜100 |
| 202 | 120 | 84 | ˜100 |

### Example 9

### Kinetic Resolution of (R,S)-naproxen by Enzymatic Esterification using Carica papaya Lipase

To an anhydrous isooctane were added Racemic (R,S)-naproxen and n-propanol to a concentration of 0.45 mM and 15 mM, respectively.

Aliquots (15 mL) of the thus-obtained solution containing racemic (R,S)-naproxen and n-propanol were added with partially purified Carica papaya lipase (PCPL, 75 mg). The resultant mixtures were allowed to react with stirring at 45° C. for 168 hrs. Thereafter, aliquots (200 μL) of samples were taken and subjected to the HPLC analysis as described in the above Example 1.

The time-course variations of the conversion rate of (S)-naproxen (expressed as X_{S}), the conversion rate of (R)-naproxen (expressed as X_{R}), the conversion rate of racemic (R,S)-naproxen at t time (expressed as Xₜ), the optical purity of the product (expressed as eeₚ), and the E value were calculated according to the descriptions set forth in the above Example 1.

The experimental data collected from this example were summarized in Table 5.

### Example 10

### Kinetic Resolution of (R,S)-2-(4-chlorophenoxy)propionic acid by Enzymatic Esterification using Carica papaya Lipase

To an anhydrous isooctane were added racemic (R,S)-2-(4-chlorophenoxy)propionic acid and a selected alcohol (n-propanol, n-butanol, n-hexanol or trimethylsilyl methanol) to a concentration of 1.5 mM and 15 mM, respectively.

Aliquots (3 mL) of the thus-obtained solution containing racemic (R,S)-2-(4-chlorophenoxy)propionic acid and the selected alcohol were added with partially purified Carica papaya lipase (PCPL, 3 mg). The resultant mixtures were allowed to react with stirring at 45° C. for a predetermined period of time. Thereafter, aliquots (200 μL) of samples were taken and subjected to the HPLC analysis as described in the above Example 6.

The time-course variations of the conversion rate of (S)-2-(4-chlorophenoxy)propionic acid (expressed as X_{S}), the conversion rate of (R)-2-(4-chlorophenoxy)propionic acid (expressed as X_{R}), the conversion rate of racemic (R,S)-2-(4-chlorophenoxy)propionic acid at t time (expressed as Xₜ), the optical purity of the product (expressed as eeₚ) and the E value were calculated according to the descriptions set forth in the above Example 1.

The experimental data collected from this example were summarized in Table 5. It can be seen that Carica papaya lipase catalyzed the esterification of (R)-2-(4-chlorophenoxy)propionic acid instead of (S)-2-(4chlorophenoxy)propionic acid.

TABLE 5

| Exam- | | Time | X_{S} or X_{R} | Xₜ | eeₚ | |
|---|---|---|---|---|---|---|
| ple | Alcohol | (hrs) | (%) | (%) | (%) | E |
| 9 | n-propanol | 168 | X_{S} = 72.0 | 36.0 | −100 | >>100 |
| 10 | n-propanol | 3.64 | X_{R} = 32.9 | 17.0 | 93.6 | 39 |
| | n-butanol | 3.69 | X_{R} = 55.1 | 28.7 | 92.0 | 40 |
| | n-hexanol | 3.38 | X_{R} = 44.6 | 24.8 | 80.2 | 12 |
| | Trimethylsilyl | 3.43 | X_{R} = 55.9 | 27.0 | 95.9 | 65 |
| | methanol | | | | | |

### CONCLUSION

It is clear from the experimental results of the above examples that Carica papaya lipase, either the commercially available crude papain or a partially purified product thereof, can be used in the enzymatic resolution of a mixture of R- and S-enantiomers of an α-substituted carboxylic acid or an ester or thioester thereof conducted in a variety of solvent systems, such as an anhydrous or water-saturated organic solvent system, giving a high yield and high conversion of an optically pure product as desired. In addition, most of the E values obtained in the above examples are greater than 30 and even over 100, indicating that Carica papaya lipase is a highly reactive biocatalyst in activating the enzymatic resolution of an α-substituted carboxylic acid or an ester or thioester thereof. The addition of an organic base during the enzymatic resolution of an α-substituted carboxylic acid or an ester or thioester thereof by Carica papaya lipase further assists in increasing the optical purity of the product to an extent reaching 100%.

All patents and literature references cited in the present specification are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claims.

1. A process for enzymatically resolving a mixture of R- and S-enantiomers of an α-substituted carboxylic acid or an ester or thioester thereof of formula (I): 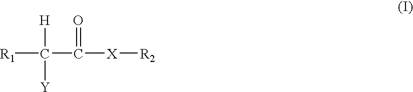
wherein
X represents —O— or —S—;
Y is a halogen or a methyl group;
R₁ represents: a straight-chain or branched saturated or unsaturated C₁-C₂₀ aliphatic group optionally substituted with one to three substituents selected from the group consisting of halo, amino, cyano, hydroxy, —SH, —COOH, —CF₃, —OCF₃, —SCF₃, —CONH₂, a C₁-C₆ alkoxy group and an aryl group; an aryl group, an aryloxy group or a C₃-C₁₂ heterocyclic group containing one to three heteroatoms selected from O, S and N, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N; and
R₂ represents: H; a straight-chain or branched saturated or unsaturated C₁-C₁₂ aliphatic group optionally substituted with one to three substituents selected from the group consisting of halo, amino, cyano, hydroxy, —CF₃, —OCF₃, —SCF₃, —Si(CH₃)₃, a C₁-C₄ alkyloxy group, a C₁-C₄ alkylthio group, an aryl group, vinyl and a 2-alkenyl group having 3 to 12 carbon atoms; an aryl group or a C₃-C₁₂ heterocyclic group containing one to three heteroatoms selected from O, S and N, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N;
with the proviso that Y and R₁ cannot be methyl at the same time; the process comprising subjecting the mixture of R- and S-enantiomers of the α-substituted carboxylic acid or ester or thioaster thereof of formula (I) to an enzymatic resolution catalyzed by a Carica papaya lipase in a liquid phase.

2. The process of claim 1, wherein the liquid phase comprises a solvent system selected from an aqueous solution, an anhydrous organic solvent, an organic solvent saturated with water, and combinations thereof forming a biphasic solution.

3. The process of claim 1, wherein the liquid phase comprises an organic solvent selected from isooctane, heptane, hexane, cyclohexane, pentane, decane, toluene, benzene, carbon tetrachloride, t-butanol, t-pentanol, isopropyl ether, methyl t-butyl ether, methyl isobutyl ether, and combinations thereof.

4. The process of claim 1, wherein the mixture is a racemic mixture of the α-substituted carboxylic acid or ester or thioester thereof of formula (I).

5. The process of claim 1, wherein the Carica papaya lipase is prepared from a latex exudate of a plant of Carica papaya.

6. The process of claim 1, wherein the enzymatic resolution of the mixture by the Carica papaya lipase is conducted in a liquid phase comprising an organic solvent in combination with an organic base.

7. The process of claim 6, wherein the organic solvent is selected from isooctane, heptane, hexane, cyclohexane, pentane, decane, toluene, benzene, carbon tetrachloride, t-butanol, t-pentanol, isopropyl ether, methyl t-butyl ether, methyl isobutyl ether, and combinations thereof.

8. The process of claim 6, wherein the organic base is selected from the group consisting of tertiary amines, amidines, guanidines, phosphazene bases, and combinations thereof.

9. The process of claim 8, wherein the organic base is selected from the group consisting of triethylamine, tributylamine, trioctylamine, 7-methyl-1,5,7-triazabicyclo[4,4,0]dec-5-ene, 1,8-diazabicyclo [5,4,0] undec-7-ene, 1,4-diazabicyclo [2.2.2] octane, t-butylimino-tris(pyrrolidino)phosphorane, t-butylimino-tris(dimethylamino)phosphorane, 1-t-butyl-4,4,4-tris(dimethylamino)-2,2-bis[tris(dimethylamino)-phosphoranylidenamino]-2λ⁵4λ⁵-catenadi(phosphazene), diethylaminomethyl-polystyrene, and combinations thereof.

10. The process of claim 8, wherein the organic base is carried on a support selected from an organic support and an inorganic support.

11. The process of claim 8, wherein the organic base is carried on an anion-exchange resin.

12. The process of claim 1, wherein the enzymatic resolution of the mixture by the Carica papaya lipase is conducted at a temperature ranging from 20° C. to 90° C.

13. The process of claim 1, wherein the mixture comprises R- and S-enantiomers of an α-substituted carboxylic acid ester or thioester of formula (I), and wherein the enzymatic resolution of the mixture by the Carica papaya lipase is conducted in a liquid phase comprising a solvent system selected from an aqueous solution, a water-saturated organic solvent and combinations thereof forming a biphasic solution, such that either R-form or S-form of the α-substituted carboxylic acid ester or thioester of formula (I) is enantioselectively hydrolyzed by the Carica papaya lipase.

14. The process of claim 13, wherein the α-substituted carboxylic acid ester or thioester of formula (I) is at least any one of the following compounds: an ethyl, propyl, butyl, hexyl, phenyl or tritluoroethyl ester of naproxen, fenoprofen, ibuprofen, ketoprofen, suprofen, flurbiprofen, 2-phenyl propionic acid, 2-(4-chlorophenoxy)propionic acid or 2-chloro-2-phenylacetic acid; an ethyl, propyl, butyl, hexyl, phenyl or trifluoroethyl thioester of naproxen, fenoprofen, ibuprofen, ketoprofen, suprofen, flurbiprofen, 2-phenyl propionic acid, 2-(4-chlorophenoxy)propionic acid or 2-chloro-2-phenylacetic acid; and diclofog methyl ester.

15. The process of claim 13, wherein the liquid phase further comprises an organic base selected from the group consisting of tertiary amines, amidines, guanidines, phosphazene bases, and combinations thereof.

16. The process of claim 15, wherein the organic base is selected from the group consisting of triethylamine, tributylamine, trioctylamine, 7-methyl-1,5,7-triazabicyclo[4,4,0]dec-5-ene, 1,8-diazabicyclo [5,4,0] undec-7-ene, 1,4-diazabicyclo [2.2.2]octane, t-butylimino-tris(pyrrolidino)phosphorane, t-butylimino-tris(dimethylamino)phosphorane, 1-t-butyl-4,4,4-tris(dimethylamino)-2,2-bis [tris(dimethylamino)-phosphoranylidenamino]-2λ⁵,4λ⁵-catenadi(phosphazene), diethylaminomethyl-polystyrene, and combinations thereof.

17. The process of claim 1, wherein the mixture comprises R- and S-enantiomers of an α-substituted carboxylic acid ester or thioester of formula (I), and wherein the enzymatic resolution of the mixture by the Carica papaya lipase is conducted in a liquid phase comprising an anhydrous organic solvent in combination with an alcohol, such that either R-form or S-form of the α-substituted carboxylic acid ester or thioester of formula (I) is enantioselectively transesterified by the Carica papaya lipase using said alcohol.

18. The process of claim 17, wherein the α-substituted carboxylic acid ester of formula (I) is at least any one of the following compounds: an ethyl, propyl, butyl, hexyl, phenyl or trifluoroethyl ester of naproxen, fenoprofen, ibuprofen, ketoprofen, suprofen, flurbiprofen, 2-phenyl propionic acid, 2-(4-chlorophenoxy)propionic acid or 2-chloro-2-phenylacetic acid; an ethyl, propyl, butyl, hexyl, phenyl or trifluoroethyl thioester of naproxen, fenoprofen, ibuprofen, ketoprofen, suprofen, flurbiprofen, 2-phenyl propionic acid, 2-(4-chlorophenoxy)propionic acid or 2-chloro-2-phenylacetic acid; and diolofog methyl ester.

19. The process of claim 17, wherein the alcohol used in the enzymatic resolution of the mixture by the Carica papaya lipase is of formula ROH, wherein R differs from R₂ and represents: a straight-chain or branched saturated or unsaturated C₁-C₁₂ aliphatic group optionally substituted with one to three substituents selected from the group consisting of halo, amino, cyano, hydroxy, —CF₃, —OCF₃, —SCF₃, —Si(CH₃)₃, a C₁-C₄ alkyloxy group, a C₁-C₄ alkylthio group, an aryl group, vinyl and a 2-alkenyl group having 3 to 12 carbon atoms; an aryl group or a C₃-C₁₂ heterocyclic group containing one to three heteroatoms selected from O, S and N, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N.

20. The process of claim 19, wherein the alcohol is selected from the group consisting of propanol, butanol, hexanol, trimethylsilyl methanol, and 2-N-morpholinoethanol.

21. The process of claim 1, wherein the mixture comprises R- and S-enantiomers of an α-substituted carboxylic acid of formula (I), and wherein the enzymatic resolution of the mixture by the Carica papaya lipase is conducted in a liquid phase comprising an anhydrous organic solvent in combination with an alcohol, such that either R-form or S-form of the α-substituted carboxylic acid of formula (I) is enantioselectively esterified by the Carica papaya lipase using said alcohol.

22. The process of claim 21, wherein the α-substituted carboxylic acid of formula (I) is at least any one of the following compounds: naproxen, fenoprofen, ibuprofen, ketoprofen, suprofen, flurbiprofen, 2-phenyl propionic acid, diclofog, 2-(4-chlorophenoxy)propionic acid and 2-chloro-2-phenylacetic acid.

23. The process of claim 21, wherein the alcohol used in the enzymatic resolution of the mixture by the Carica papaya lipase is of formula ROH, wherein R differs from R₂ and represents: a straight-chain or branched saturated or unsaturated C₁-C₁₂ aliphatic group optionally substituted with one to three substituents selected from the group consisting of halo, amino, cyano, hydroxy, —CF₃, —OCF₃, —SCF₃, —Si(CH₃)₃, a C₁-C₄ alkyloxy group, a C₁-C₄ alkylthio group, an aryl group, vinyl and a 2-alkenyl group having 3 to 12 carbon atoms; an aryl group or a C₃-C₁₂ heterocyclic group containing one to three heteroatoms selected from O, S and N, each group being optionally substituted with one to three substituents selected from the group consisting of halo, amino, cyano, hydroxy, —SH, —COOH, —CF₃, —OCF₃, —SCF₃, —CONH₂, a C₁-C₆ alkoxy group, an aryl group and a C₃-C₁₂ heterocyclic group containing one to three heteroatoms selected from O, S and N.

24. The process of claim 23, wherein the alcohol is selected from the group consisting of propanol, butanol, hexanol, trimethylsilyl methanol, and 2-N-morpholinoethanol.
